(19) United States
(12) Patent Application Publication
(10) Pub. No.: US 2026/0294236 A1
(43) Pub. Date: Oct. 1, 2026

(54) Method and Device for Measuring Optical Quality of the Human Eye and Its Crystalline Lens

(71) Applicant: Tracey Technologies, Corp., Houston, TX (US)

(72) Inventors: Vasyl Molebny, Houston, TX (US); Joe Wakil, Houston, TX (US); Sridhar Madala, Houston, TX (US); Sergiy Molebny, Kiev (UA); Oleksandr Lutsenko, Chernihiv (UA); Ioannis Pallikaris, Maleviziou, Crete (GR)

(73) Assignee: Tracey Technologies, Corp., Houston, TX (US)

(21) Appl. No.: 19/480,862

(22) PCT Filed: Apr. 30, 2024
(86) PCT No.: PCT/US2024/026954

§ 371 Date: Nov. 3, 2025

Related U.S. Application Data
(60) Provisional application No. 63/499,344, filed on May 1, 2023.

(51) Int. Cl. A61B 3/103; A61B 3/10; A61B 3/14

(52) U.S. Cl. CPC A61B 3/103; A61B 3/1015; A61B 3/14

(57) ABSTRACT

Provided herein are methods and systems for measuring an optical quality of an eye that is based on laser ray tracing. Generally, the method utilizes steps of probing the human eye sequentially in time with one or a plurality of laser beams in a set of points within an aperture of the eye, detecting the projections of the laser beam reflected and scattered from the retina and processing the detected images of the laser spots. Components of the system include, but are not limited to, a laser with a two-axis acousto-optical deflector and with a two-output generator of digitally synthesized sinusoidal voltages in one embodiment, a collimating optical system, a conjugating telescopic system, a polarizing beam splitter, and first and second photosensitive detectors each of which are in operable communication with a processing unit. Another embodiment utilizes a multi-laser block which does not require laser beam scanning.

### CROSS-REFERENCE TO RELATED APPLICATIONS

This international patent application claims benefit of priority under 35 U.S.C. § 119(e) of provisional patent application U.S. Ser. No. 63/499,344, filed May 1, 2023, the entirety of which is hereby incorporated in its entirety.

### BACKGROUND OF THE INVENTION

### Field of the Invention

The field of present invention is ophthalmic instrumentation used for eye examination. Particularly, the present invention relates to ophthalmic examination instruments that measure the quality of vision, especially related to the optical properties of the crystalline lens defining its quality that are principal in decision making on the surgery of lens replacement.

### Description of the Related Art

The quality of human vision is a function of many parameters of external and internal origin. External factors include illumination, its level from scotopic to photopic, its spectrum, state of the atmosphere, etc. Internal components include the optical and physiological properties of the optical system, health of the retinal photoreceptors, the state of the nerve communication system, and processing abilities of brain. The interests of refractive and cataract surgery are concentrated mainly on the optical system, particularly, on its components—cornea and crystalline lens when they are the objects of surgery.

Initially, surgical technologies of vision correction were based on measurement of the shape of the cornea, derivation of wave front errors from the shape under certain suggestions, and correcting those errors using laser technologies, like laser ablation of corneal tissue, the laser in situ keratomileusis (LASIK) being one of them. Shortly, the principles were proposed of measuring the wave front errors (aberrations) of the total eye, providing more accurate information on the amount of tissue to be ablated from the corneal layers.

Several methods of measurement of the wave front errors of the human eye are known. Their detailed analysis is given in the Handbook of Visual Optics, P. Artal, Ed., Volume 2, CRC Press, 2017 (Chapter 2. V. Molebny, Wavefront sensors, pp. 17-36).

One method of measurement of the wave front errors of the human eye is based on the use of a Hartmann-Shack sensor (J. Liang et al. Objective measurement of wave aberrations of the human eye with the use of a Hartmann-Shack wave-front sensor. Journal of the Optical Society of America, 1994, Vol. 11, No. 7, pp. 1949-1957). According to this method, a thin laser beam is directed to the retina in parallel to the visual axis of the eye, its spot on the retina is imaged by a matrix of micro lenses on the photosensitive surface of the video camera. From the received data, a map of wavefront errors is reconstructed.

Simultaneous projection of a regular structure of light on the retina is also used for reconstruction of aberrations from the distorted image of the projected structure. (P. Mierdel et al. in Ocular optical aberrometer for clinical use. Journal of Biomedical Optics, 2001, Vol. 6, No. 2, pp. 200-204).

Widely used is the laser ray tracing, time-sequential approach of V. Molebny et al. (Ukraine patent No. 46833, U.S. Pat. No. 6,932,475, etc.), according to which the eye is probed point by point with a laser beam sequentially in time, its projection on the retina is detected, coordinates of the projection spots are measured, and the map of wave front errors is reconstructed. Based on the results of the measurements, other characteristics like modulation transfer function, point spread function, etc. are calculated.

None of the above-mentioned technologies provides a separate data on the wave front errors of the crystalline lens itself, cleaned from the errors inserted by the cornea. To get the refractive characteristics of the crystalline lens, corneal wave front is to be reconstructed first from its topography that is measured by projecting a regular light structure on the cornea. The wave front errors of the crystalline lens are derived by subtracting the corneal wave front from the wave front of the optical system of the eye.

A review of these techniques is given in Corneal Topography in the Wavefront Era, M. Wang, Ed., Slack Inc., Thorofare, N.J., 2006. The main complaint in acquiring a wave front map of the crystalline lens is due to non-synchronous/non-simultaneous measurements of the to be transformed values. An effort to resolve this problem was undertaken by V. Molebny, et al. (Method and device for synchronous mapping of the total refraction non-homogeneity of the eye and its refractive components. U.S. Pat. No. 6,409,345) with the math for the analysis described in Ophthalmic and Physiological Optics, 2009, Vol. 29, pp. 330-337 (V. Molebny et al. “Damped least-squares approach for point-source corneal topography”) that enabled significant increasing of the accuracy.

However, even under these conditions, in the case of a complex topography of the cornea, e. g., with steep profile variations, measurement based on indirect calculation of the refractive characteristics of the lens may lead to incorrect results taking also into account that the reconstructed wave fronts are the result of approximation that may result in the loss of essential information on the spatial high-frequency components of the refractive inhomogeneity of the lens, that are the principal factors in the lens refractive dysfunction.

A solution known from the Patent of Ukraine 122709 (V. Molebny. “Method of synchronous ray tracing measurement of the refractive errors of the eye and of the refraction non-homogeneity of its crystalline lens”) suggests penetration into the eye of two laser beams in the same point of the cornea but being tilted by different angles. Unfortunately, it lacks the calibrated values of the refractive non-homogeneity of the lens that is of critical significance for planning the surgery.

Another component of the lens optical quality originates from its transparence, or its inverse value—opacity described as light extinction. The opacity depends on two main phenomena leading to loss of light in the eye—light absorption and light scatter. In a simple way, the scatter can be evaluated with a slit lamp by observing light scattering in the crystalline lens. With the laser light involved in the wave front measurements, the attempts were made to measure the opacity as a side opportunity, evaluating it by measuring the degree of the total laser light coming back from the eye and suggesting that the more light returns back, the more transparent the lens is. Actually, this suggestion is not correct for the light propagating in a partially transparent media. As it follows from the light transport theory (R. Carminati, J. C. Schotland. Principles of scattering and transport of light. Cambridge University Press, 2021), in scattering media, the amount of light is not only diminishing in the process of propagation forth and back due to absorption and scatter, but also increasing on its path back by certain amount due to random, multiple, and omnidirectional character of scattering. The balance between the diminished and increased amount of light is not known, neither during, nor after the process of measurement by any of the known techniques.

Therefore, a need is recognized to redeem the techniques of determining the optical quality of the crystalline lens of the human eye based on specific features of the laser ray tracing approach.

### SUMMARY OF THE INVENTION

The present invention is based on the specific features of the laser ray tracing technique considering reflection and scatter components of laser light (both of them) in the process of its propagation and transformations in the eye. The traditionally used scatter component coming back from the eye through the entire aperture of the eye is the only source of information for the reconstruction of the eye's refractive parameters. The reflection component of the narrow laser beam propagating in eye media in the outward direction delivers extra information about the refractive parameters and energy losses. Combining these two sources of information creates an opportunity to get the estimate of the quality of the crystalline lens.

The method includes the procedures of probing the eye sequentially in time with a laser beam in a set of points within the aperture of the eye, detecting the image of the laser spots on the retina with a photosensitive detector, processing the detected signals, and reconstructing the distribution of the wave front errors.

Preliminary probing in parallel to the optical axis provides the information on the defocus component. Then, a linearly polarized laser beam is projected into the eye at an angle compensating for the measured defocus component, and the detection is made in two image planes; the first is conjugated with the object plane of the retina, and the second image plane is conjugated with the object plane inside the crystalline lens.

In the light coming from the eye to the first image plane, the light component is suppressed having the same polarization with the probing laser beam, that corresponds to the light reflected from the retina. No suppression of the reflected component is made in the second image plane.

In the first image plane, the coordinates of the spots of laser light in the plane of the retina scattered in the tissues of the retina are measured by the photosensitive detector, and, after the processing, the two-dimensional distribution of the wave front errors is reconstructed.

In the second image plane, signal levels are measured of each of two spots imaged on the photosensitive detector, where the first spot is produced by the reflection of the laser beam from the reflecting surface of the retina and having passed through the crystalline lens in the outward direction, and the second spot is the image of the scattering trace of the outward passing laser beam in the crystalline lens. As an option, the measurement signal levels of each of the spots is provided with spatial selection of zones of their location. Still another option comprises the measurement in an annular zone, whose internal size is restricted by the zone of Purkinje reflections.

A ratio of the signal level of the second spot to the signal level of the first spot in the zone of emergence of the reflected laser beam from the eye is calculated for all zones of beam emergence and is represented as a two-dimensional distribution of scatter in the crystalline lens.

In one version of the method provided herein, the information on the two-dimensional distribution of scatter in the crystalline lens is combined with the information on the two-dimensional distribution of wave front errors in the crystalline lens resulting in the degradation of the modulation transfer function. The optical quality of the crystalline lens scaled in a dimensionless index is calculated from this degraded modulation transfer function.

Another version of the proposed method comprises the following procedures.

In the second image plane, coordinates are measured of the reflected laser beam for all probing points. The difference of measured coordinates is calculated for pairs of symmetrical probing points (a probing point with coordinates (x, y) is symmetrical to the probing point with coordinates (−x, −y)). The differential wave front errors are reconstructed from the difference of measured coordinates, and the modulation transfer function is calculated. Combining this with the information on two-dimensional distribution of scatter in the crystalline lens defines the optical quality of the crystalline lens scaled in a dimensionless index.

A device for measurement of the optical quality of the crystalline lens of the human eye contains a laser, a two-axis acousto-optical deflector driven by a pair of drivers configured to deflect the laser beam in orthogonal directions, the drivers are fed from the generator of digitally synthesized sinusoidal voltages. A collimating optical system is placed at the exit of the deflector with its front focus in the center of the beam deflection. The farther path of the beam is through a conjugating telescopic system having at least one electrically controlled element with variable optical power compensating the ametropy of the eye. A polarizing beam splitter is placed between the collimating optical system and the conjugating telescopic system. A first photosensitive detector is placed in the path of the laser light reflected by the polarizing beam splitter. The conjugating telescopic system has a feature to optically conjugate the plane of the retina with the plane of detection. At the same time, it corrects the tilt of laser beams, thus compensating the ametropy of the eye.

A second photosensitive detector is optically conjugated with the plane inside the crystalline lens orthogonal to the optical axis. As an option, both photosensitive detectors are featured with a restricted read-off zone of the photosensitive area. A processing unit controls the operation of the laser, of the generator of digitally synthesized sinusoidal voltages, of the conjugating telescopic system, and of the photosensitive detectors, including the restricted read-off zone.

An alternative device comprises a multi-laser block containing a plurality of lasers, each one configured to independently emit a laser beam where the optical axis of each laser is oriented to cross the optical axis of the device in a single point, a collimating optical system and the other components of the device. The configuration and functioning thereof are similar to the device with a single laser and the acousto-optical deflectors.

### BRIEF DESCRIPTION OF THE DRAWINGS

So that the above-recited features of the invention are to be understood in detail, more particular descriptions of the invention briefly summarized above are illustrated in the appended drawings. These drawings form a part of the specification. However, that the appended drawings illustrate preferred embodiments of the invention, they are not to be considered limiting in their scope.

FIG. 1 is a functional schematic of the device for measurement of the optical quality of the crystalline lens of the human eye.

FIG. 2 illustrates time-sequential positions of the laser beam in the aperture of the eye and the corresponding driving frequencies applied to the acousto-optical crystals for X and Y directions.

FIG. 3A shows the path of the laser beam in an emmetropic eye. Forward and backward propagating laser beam is shown as pattern filled. Laser light scattered by the retina is designated by dotted tracks.

FIG. 3B illustrates how part of the light is reflected from the retina (the case of an emmetropic eye) and how another part is scattered.

FIG. 4A demonstrates the path of the laser light scattered by the retina with suppression of the reflected component by a polarizing beam splitter (the vertical component passes through it, the horizontal one is reflected to the detector). The detector plane and the retina are in the conjugated position (telescope is not shown).

FIG. 4B shows the exclusion of the reflected component from its farther propagation to the detector.

FIG. 4C shows detector signal corresponding to the light intensity distribution in the detector plane.

FIGS. 5A-5B show, with the same designation as in FIG. 3A, the laser beam reflection from the surface of the retina, and scatter in the tissue of the retina in a hyperopic eye with a probing laser beam parallel to the optical axis (FIG. 5A) and with a tilted probing laser beam compensating for the Zernike defocus component (FIG. 5B).

FIG. 5C illustrates how part of the light is reflected from the retina and how another part is scattered in a hyperopic eye.

FIGS. 6A-6B illustrate the same processes as in FIGS. 5A-5B, for a myopic eye.

FIG. 6C illustrates how part of the light is reflected from the retina and how another part is scattered in a myopic eye.

FIG. 7 shows the spot of a laser projection on retina as it is imaged in the detector plane, with its profiles along X and Y coordinates, read out from the photosensitive matrix. The defocus component of eye aberrations is not compensated.

FIG. 8 shows the image of the same spot as in FIG. 7, after compensation of the Zernike defocus component by tilt of the laser beam and conjugation of the detector plane with the plane of retina. The spot with a smaller size has been shifted to the center of the matrix, i.e., to the optical axis.

FIG. 9 is a retina spot diagram acquired with a probing beam parallel to the optical axis.

FIG. 10 is a retina spot diagram acquired with a probing beam tilted to compensate the Zernike defocus component of the reconstructed wave front.

FIG. 11 illustrates the path of the probing laser beam in the crystalline lens of a hyperopic eye: beam a—parallel to the optical axis, beam b—tilted by a given angle γ.

FIG. 12 illustrates the path of the probing laser beam in the crystalline lens of a myopic eye: beam a—parallel to the optical axis, beam b—tilted by a given angle μ.

FIG. 13 are retinal spot diagrams—projections of probing laser beams a and b, and their coordinate difference (Δxᵢ, Δyᵢ), . . . (Δxᵢ₊ₙ, Δyᵢ₊ₙ).

FIG. 14 is a differential wave front diagram.

FIGS. 15A-15B demonstrate the path of the laser beam reflected from the retina and the component of its scatter in the crystalline lens on its way back from the eye (the case with the emmetropic eye). The detector plane is conjugated with the plane in the crystalline lens. The inset a explains the process of scatter in the crystalline lens.

FIGS. 15C-15E show the profiles, corresponding to different phenomena, of a defocused image of scattering by the retina (FIG. 15C), an image of the scattering volume in the crystalline lens (FIG. 15D), and a projection of the laser reflex from the retina (FIG. 15E).

FIG. 16 is an image of the laser reflex from the retina acquired with the component polarized in the plane of the laser beam, with its profiles along X and Y coordinates, with the readout from a restricted zone of the photosensitive matrix.

FIG. 17 illustrates the symmetry of coordinates of the incoming and outcoming laser beams (the case with the emmetropic eye): the beam enters the eye in the point (xᵢ, yᵢ), and exits at the point in the vicinity of (−xᵢ, −yᵢ). The readout zone of the detector matrix is adjusted for the vicinity of (−xᵢ, −yᵢ).

FIG. 18 illustrates the same phenomenon as in FIG. 17 with the reversed coordinates: the beam enters the eye in the point of (−xᵢ, −yᵢ), and exits at the point in the vicinity of (xᵢ, yᵢ). The readout zone of the detector matrix is adjusted for the vicinity of (xᵢ, yᵢ).

FIG. 19 is a diagram of light scatter in the crystalline lens.

FIG. 20 is a functional schematic of a device for measurement of the optical quality of the human eye and of its crystalline lens, where the probing procedures are performed by a multi-laser block dispatched electrically without acousto-optical scanning.

### DETAILED DESCRIPTION OF THE INVENTION

As used herein, the articles “a” and “an” when used in conjunction with the term “comprising” in the claims and/or the specification, may refer to “one”, but it is also consistent with the meaning of “one or more”, “at least one”, and “one or more than one”. Some embodiments of the invention may consist of or consist essentially of one or more elements, components, method steps, and/or methods of the invention.

As used herein, the term “or” in the claims refers to “and/or” unless explicitly indicated to refer to alternatives only or the alternatives are mutually exclusive, although the disclosure supports a definition that refers to only alternatives and “and/or”

As used herein, the terms “comprise” and “comprising” are used in the inclusive, open sense, meaning that additional elements may be included.

As used herein, the terms “consists of” and “consisting of” are used in the exclusive, closed sense, meaning that additional elements may not be included.

As used herein, the term “includes” or “including” is used herein to mean “including, but not limited to”. The terms “includes, “including” and “including, but not limited to” are used interchangeably

In one embodiment of the present invention, there is provided a method for measuring an optical quality of a human eye based on laser ray tracing, the method comprising probing the human eye sequentially in time with a laser beam in a set of points within an aperture of the eye, detecting the projections of the laser beam reflected and scattered from the retina; and processing the detected images of the laser spots.

Further to this embodiment, the method comprises calculating a Strehl ratio for each probing point separately, said calculation made on signal intensity distributions in detected images of the laser spots of laser projections comprising their point spread functions in a status of a compensated defocus of the projected laser beam and of the detected images of said laser spots; reconstructing a map of the optical quality of the eye with the values of said optical quality in each probing point that is a function of the Strehl ratio; and calculating a total quality of the eye as a ratio of a number of probing points with a quality higher than the established threshold to the total number of the probing points.

In another embodiment of the present invention, there is provided a method for measuring an optical quality of a human eye based on laser ray tracing, comprising probing the human eye sequentially in time with a linearly polarized laser beam in a set of points within the aperture of the eye; detecting images of laser spots on a retina with a photosensitive detector; processing detected signals; and reconstructing a distribution of wave front errors.

Further to this embodiment, the method comprises calculating a defocus component from the reconstructed distribution of wave front errors; probing the human eye with the linearly polarized laser beam projected therein at an angle compensating for the defocus component; selecting an orthogonally polarized component of light coming from the human eye, detecting the orthogonally polarized component in a detection plane conjugated with the retina, measuring a shape and coordinates of a detected image of a spot of the laser projection on the retina; calculating a Strehl ratio in each probing point separately for the point spread function described by a measured shape of the spot image of the projected laser beam, reconstructing a map of the optical quality of the human eye with values of the optical quality in each probing point that is the function of the Strehl ratio, and calculating a total quality of the eye represented by a ratio of the number of probing points with the quality higher than an established threshold to the total number of the probing points.

In yet another embodiment of the present invention, there is provided a method for measuring an optical quality of a crystalline lens of an eye based on laser ray tracing, comprising probing the eye sequentially in time with a laser beam in a set of points within an aperture of the eye; detecting images of laser spots on a retina with a photosensitive detector, processing the detected signals; reconstructing the distribution of the wave front errors; and calculating the modulation transfer function.

Further to this embodiment, the method comprises calculating a defocus component from the reconstructed distribution of the wave front errors; probing the eye in a set of probing points over the aperture thereof with a linearly polarized laser beam projected into the eye at a first angle that is an angle compensating for a defocus component; selecting an orthogonally polarized component of a light coming from the eye; detecting said orthogonally polarized component in a detection plane conjugated with the retina; measuring coordinates of the detected image of a spot of a laser projection on the retina; probing the eye in the same set of probing points over the aperture with the linearly polarized laser beam projected in the eye at a second angle differing from the first angle by a normalization value; selecting the orthogonally polarized component of the light coming from the eye, detecting said orthogonally polarized component in the detection plane conjugated with the retina; measuring coordinates of the detected image of a spot of the laser projection on the retina; calculating a difference between coordinates of the detected spot images at the first angle of the probing laser beam and the second angle of the probing laser beam in the same probing points; reconstructing a two-dimensional distribution of the wave front difference; and calculating a ratio of an area of a surface of the two-dimensional distribution having a wave front difference lower than an established threshold of a total area of the surface of said two-dimensional distribution. In both embodiments, the optical quality of the crystalline lens is a function of the calculated ratio.

In yet another embodiment of the present invention, there is provided a method for measuring an optical quality of a crystalline lens of an eye, based on laser ray tracing, the method comprising probing the eye sequentially in time with a laser beam in a set of points within an aperture of the eye; detecting images of the laser spots on a retina with a photosensitive detector; processing the detected signals; and reconstructing a distribution of wave front errors; wherein a preliminary probing of the eye in parallel to an optical axis thereof is provided and a defocus component is derived from the reconstructed wave front errors; said laser beam is linearly polarized and is projected in the eye at an angle compensating for said defocus component; a first plane of detection is provided and is conjugated with a plane of a retina and a second plane of detection is conjugated with a plane inside the crystalline lens orthogonal to the optical axis; the light coming from the eye is detected separately for each of a first polarized component and a second polarized component, said first polarized component having a plane of polarization orthogonal to the polarization of the probing laser beam, the second polarized component being in the plane of polarization of said probing laser beam; in a first plane of conjugation, coordinates are measured of the spot imaged on a two-dimensionally photosensitive detector for the first polarization component, corresponding to the laser beam scattered in the retina, and the two-dimensional distribution of the wave front errors is reconstructed; in a second plane of conjugation, signal levels are measured of each of a first spot and a second spot imaged on the two-dimensionally photosensitive detector for the second polarization component, said first spot produced by the reflection of the laser beam from the retina and having passed through the crystalline lens in an outward direction, and said second spot being an image of a scattering trace of the outward passing laser beam in the crystalline lens; a two-dimensional distribution of scatter in the crystalline lens is reconstructed as a ratio of a signal level of the second spot to a signal level of the first spot, and its dimensionless index is calculated as an averaged value of said ratios; and the optical quality of the crystalline lens scaled in a dimensionless index is calculated as a degradation of a modulation transfer function of the eye without a deducted corneal component, said degradation of the modulation transfer function is referenced to said two-dimensional distribution of scatter in the crystalline lens.

In this embodiment, the coordinates of the first spot imaged in the second plane of conjugation may be measured for the second polarization component, where the measurements are provided in a central zone of the two-dimensionally photosensitive detector such that signals outside the central zone are excluded from measurements, and a size of the central zone is restricted by a size of a cross-section of the reflected laser beam and a preassigned range of dispersion of spot coordinates.

Particularly, the coordinates of the first spot that are a result of ray tracing of the eye through any of the set of probing points are compared with the coordinates of another first spot that a the result of ray tracing of the eye through the probing point positioned in an aperture of the eye symmetrically with the optical axis; and a differential modulation transfer function is calculated from a difference between the compared spot coordinates, and the optical quality of the crystalline lens scaled in a dimensionless index is calculated as a degradation of the differential modulation transfer function as referenced to the two-dimensional distribution of scatter in the crystalline lens. Also, the coordinates of the second spot imaged in the second plane of conjugation are measured for the second polarization component, the measurements provided in a central zone with the coordinates of its center symmetrical with the coordinates of eye probing by the laser beam in regard to the optical axis, the signals outside the zone excluded from measurements, and a size of the zone restricted by a size of a cross-section of the reflected laser beam and a preinstalled range of the spot coordinates dispersion.

Also, the coordinates of the first spot imaged in the second plane of conjugation are measured for the second polarization component, the measurements provided in an annular zone, whose internal size is restricted by a zone of Purkinje reflections; wave front errors are calculated with deduction of the corneal component in the annular zone, and compared with the wave front errors in said annular zone calculated from the measurements of the spot coordinates imaged in the first plane of conjugation for the first polarization component; and a differential modulation transfer function is calculated for the annular zone, and the optical quality of the crystalline lens scaled in a dimensionless index is calculated as the degradation of differential modulation transfer function, being referenced to said two-dimensional distribution of scatter in the annular zone. In addition, a value of any of the signals partially or completely created by the second polarization component exceeding a preinstalled level regarding an average value, and its exclusion from calculations of the optical quality of the crystalline lens is evaluated.

In yet another embodiment of the present invention, there is provided a device for measurement of the optical quality of the crystalline lens of the human eye, comprising a laser configured to emit a laser beam of a wavelength suitable for propagation in the human eye; a two-axis acousto-optical deflector on a path of the laser beam; a two-output generator of digitally synthesized sinusoidal voltages with its outputs electrically connected to the two-axial acousto-optical deflector and configured to control the deflection of the laser beam in both orthogonal directions; a collimating optical system placed at the exit of the two-axis acousto-optical deflector with its front focus in a center of beam deflection; a conjugating telescopic system with a lens component or a mirror component and with its optical axis coincident with an optical axis of the collimating optical system; a polarizing beam splitter placed between the collimating optical system and said conjugating telescopic system; a first photosensitive detector with a coordinate measurement function placed in the path of the laser light reflected by said polarizing beam splitter with the feature of optical conjugation with the plane of the retina; a second photosensitive detector with a profile measurement function is placed through a beam splitter installed between the eye and the lens component or the mirror component of the conjugating telescope system, the profile measuring photosensitive detector is optically conjugated with the plane inside the crystalline lens orthogonal to the optical axis; and a processing unit in operable communication with each of the laser, the two-output generator of digitally synthesized sinusoidal voltages, the conjugating telescopic system, and the position sensitive detection system.

In this embodiment, the two-axial acousto-optical deflector may be operably connected to a pair of drivers, each of the pair with outputs electrically connected to the two-axial acousto-optical deflector and configured to deflect the laser beam with regard to the optical axis in orthogonal directions. Also in this embodiment the conjugating telescopic system may comprise at least one electrically controlled element with a variable optical power configured to compensate for an ametropy of the eye. In addition, the coordinate systems of deflection and detection may be coincident. Furthermore, the optical conjugation of the photosensitive plane of the coordinate measuring photosensitive detector is via the conjugating telescopic system, the optical conjugation adaptive to the optical power of the eye, the adaptation provided by at least one of the lens component or the mirror component of the conjugating telescopic system that is configured to control the optical power of both of the lens component and the mirror component via communication with said processing unit. Further still in this embodiment, the polarizing beam splitter may be configured to suppress the component of light polarized in the plane of polarization of the laser beam returning from the eye in the direction to the coordinate measuring photosensitive detector. Further still the second detector channel may be configured to read-off the information from the restricted zone of the photosensitive area, the restricted zone controlled through the communication with said processing unit.

In yet another embodiment of the present invention, there is provided a device for measurement of the optical quality of the crystalline lens of the human eye, comprising a multi-laser block containing a plurality of lasers, each of said plurality configured to emit independently a laser beam of a wavelength suitable for propagation in the human eye, an optical axis of each laser in the plurality oriented to cross an optical axis of the device in a single point; a collimating optical system placed on the optical axis of the device with its front focus in a center of beam deflection; a conjugating telescopic system with a lens component or a mirror component having an optical axis coincident with an optical axis of the collimating optical system; a polarizing beam splitter placed between the collimating optical system and the conjugating telescopic system; a first photosensitive detector with a coordinate measurement function placed in a path of laser light reflected by said polarizing beam splitter with a feature of optical conjugation with a plane of a retina in the human eye; a second photosensitive detector with a profile measurement function is placed through a beam splitter positioned between the human eye and the lens component or the mirror component of the conjugating telescope system, the profile measuring photosensitive detector is optically conjugated with the plane inside the crystalline lens orthogonal to the optical axis; and a processing unit in operable communication with each of the plurality of lasers, a two-output generator of digitally synthesized sinusoidal voltages, the conjugating telescopic system, and a position sensitive detection system.

In this embodiment the conjugating telescopic system comprises at least one electrically controlled element with a variable optical power configured to compensate for an ametropy of the eye. Also in this embodiment the optical conjugation of the photosensitive plane of said coordinate measuring photosensitive detector is via the conjugating telescopic system, said optical conjugation adaptive to the optical power of the eye, said adaptation provided by at least one of the lens component or the mirror component of the conjugating telescopic system that is configured to control the optical power of both of the lens component and the mirror component via communication with said processing unit. In addition the polarizing beam splitter may be configured to suppress the component of light polarized in the plane of polarization of the laser beam returning from the eye in the direction to the coordinate measuring photosensitive detector. Furthermore the second detector channel may be configured to read-off the information from the restricted zone of the photosensitive area, said restricted zone controlled through communication with the processing unit.

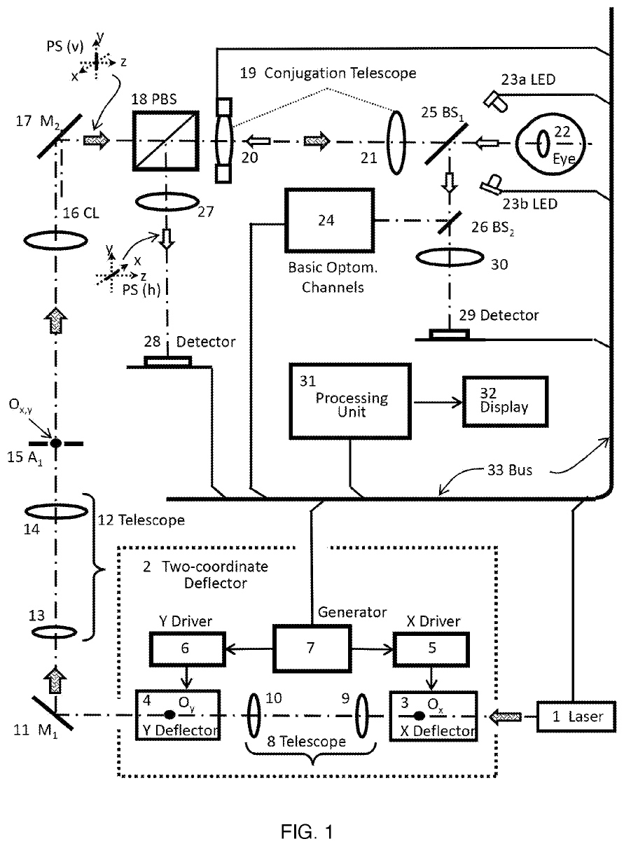

The principles of this invention include several procedures that are better understood from the functional schematic of the device of FIG. 1 illustrating all these procedures. The embodiments of this invention are not limited by the described design and sequences of functional interactions.

The device comprises a laser 1, optical and electro-optical components 2-25 with necessary connections performing the probing of the eye with the laser beam, image forming and photosensitive components 26-30, and processing and displaying means 31 and 32.

Optical and electro-optical components in alignment are: two-coordinate deflector 2, first mirror (M₁) 11, telescope 12, aperture 15, collimating lens (CL) 16, second mirror (M₂) 17, polarizing beam splitter (PBS) 18, conjugation telescope 19, and first beam splitter (BS₁) 25.

The two-coordinate deflector 2 placed in line with the laser 1 contains X deflector 3, Y deflector 4, X driver 5 with its output connected to the X deflector 3, Y driver 6 with its output connected to the Y deflector 4, and generator 7 connected to both X and Y drivers 5, 6. On the optical path between deflectors 3 and 4, a telescope 8 is placed with a unit magnification consisting of two lenses 9 and 10 with equal optical power. They are configured in a typical telescope manner: the front focus of the lens 9 coincides with the center of scanning of the X deflector 3, the back focus of the lens 10 coincides with the center of scanning of the Y deflector 4, and the back focus of the lens 9 coincides with the front focus of the lens 10. The generator 7 is controlled by a processing unit 31 through a bus 33.

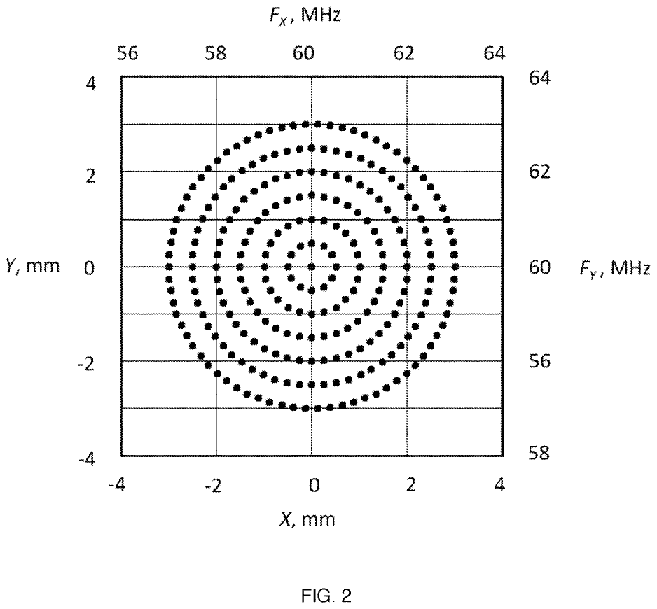

The deflection in the acousto-optical crystals of both, X and Y deflectors 3, 4, is provided by an elastic diffraction grating created in the crystals with acousto-optical effect by the harmonic voltage of a certain frequency applied to the excitation electrodes on the surface of the crystals. By way of example, in the diagram of FIG. 2, shown are the frequencies applied to the excitation electrodes resulting in probing the eye in the points with (x, y) coordinates. Sine voltages for both X and Y deflection are digitally synthesized by the generator 7 controlled by the processing unit.

The first mirror M₁ 11, placed on the path of the laser beam at the exit of the two-coordinate deflector 2, turns the optical path by 90 degrees. Telescope 12 consists of two lenses, 13, 14. Its magnification is higher than a unit, typically about 5^{x}, to magnify the deflection angle provided by the two-coordinate deflector 2. The telescope 12 is designed also in a typical telescope configuration: the front focus of the lens 13 coincides with the center of scanning of the Y deflector 4, the back focus of the lens 14 coincides with the center of the aperture A₁ 15, and the back focus of the lens 13 coincides with the front focus of the lens 14.

The next optical element on the path of the laser beam is a collimating lens (CL) 16, whose front focus coincides with the center of the aperture 15. The second mirror M₂ 17 bents the beam direction by 90 degrees. The next component is a polarizing beam splitter PBS 18. It allows the polarized laser radiation to pass farther along to the eye. The farther path is through a conjugating telescope 19 consisting of two lenses, 20 and 21. The first lens 20 is a fluidic one with a variable optical power that is electrically controlled by a processing unit 31 through the bus 33.

The eye 22 is side illuminated by several point light sources 23a LED, 23b LED, etc.). Their number is not a principal matter. Usually, there are 4 or 6. Light emitting diodes are the good candidates. Two of them, LED 23a and LED 23b are shown in the schematic of FIG. 1. The reflections of these point light sources are looked at by basic optometry channels 24 through a first beam splitter 25 (BS₁) and a second beam splitter 26 (BS₂).

There are two photosensitive detectors designed to coordinate measuring. The first detector 28 is placed on the path of the light coming out of the eye 22, which is bent by the polarizing beam splitter 18. The second detector 29 is placed on the path of the light coming out of the eye 22 after being bent by the beam splitter 25 BS₁ and having crossed the beam splitter 26 BS₂. In front of the first detector 28, a lens 27 is placed enabling the conjugation of the photosensitive plane of the first detector with the plane of retina, in combination with the conjugating telescope 19. In front of the second detector 29, a lens 30 is placed conjugating the photosensitive plane of the second detector 29 with the plane inside the crystalline lens of the eye 22.

The functions of the present invention are provided by several procedures, the first of them is the probing of the eye sequentially in time with a laser beam generated by the laser 1, in a set of points within the aperture of the eye. An example of a set of such points is given in FIG. 2. To control the positions of the points of laser probing, the two-coordinate deflector 2 is used to direct each beam at a prescribed angle with regard to the optical axis. Four optical systems participate in positioning the beams in the prescribed points of the aperture of the eye with the prescribed tilts.

The first of the optical systems is the telescope 8, translating the center of scanning Oₓ in the X direction provided by the acousto-optical crystal of the deflector 3 to the center of scanning O_{y} in the Y direction provided by the acousto-optical crystal of the deflector 4. This resolves the problem of having a single-center scanning in both directions in the two-coordinate deflector 2 consisting of two single-coordinate deflectors 3 and 4. It is important for avoiding the errors in the reconstruction of the wave front.

The role of the telescope 12 is to widen the range of the beam deflection by magnifying it, for example, from 1 degree at the exit of the deflector 2 to 5 degrees at the exit of the telescope 12. The above described design of the telescope 12 provides the translation of the centers of scanning Oₓ and O_{y} into a single center of scanning O_{x,y}. The point O_{x,y} is positioned in the center of the aperture 15 playing the role of a spatial filter selecting the first order of refraction in both directions of deflection. It coincides with the front focus of the collimating lens 16, thus providing the orientation of each beam exiting the collimating lens, parallel to the optical axis. The orientation of the beams parallel to the optical axis is kept by the conjugating telescope 19 at the start of measurements. In further measurements, it is changed to compensate for the defocus component of the reconstructed wave front if the eye is ametropic.

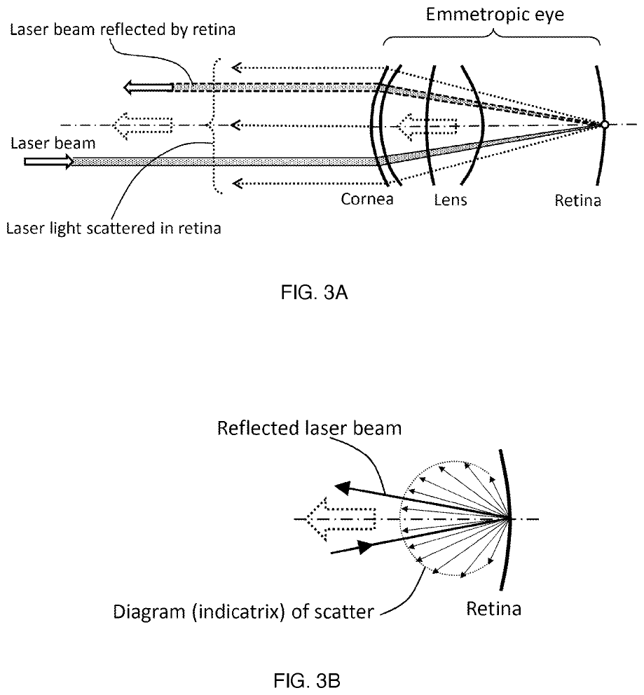

In each point of eye probing, the laser beam crosses the cornea with all its layers, the anterior chamber, the crystalline lens with its anterior and posterior surfaces, the posterior chamber, and finally reaches the retina (FIG. 3A). Each surface influences the propagation of the beam by reflecting and tilting the beam. The media of the eye are not ideally transparent, so they absorb a part of the light, and scatter some of the other part of it. The main player in light scattering in the eye is the crystalline lens, especially with aging. FIG. 3B shows the two-fold role of the retina. Part of the light is reflected; another part is scattered. The part of the light absorbed by the tissues of the retina is not taken into account. The eye in the example of FIG. 3A is emmetropic, the reflected beam exits the eye symmetrically to the probing beam with regard to the optical axis. The bunch of scattered rays exits the eye the same in parallel to the optical axis.

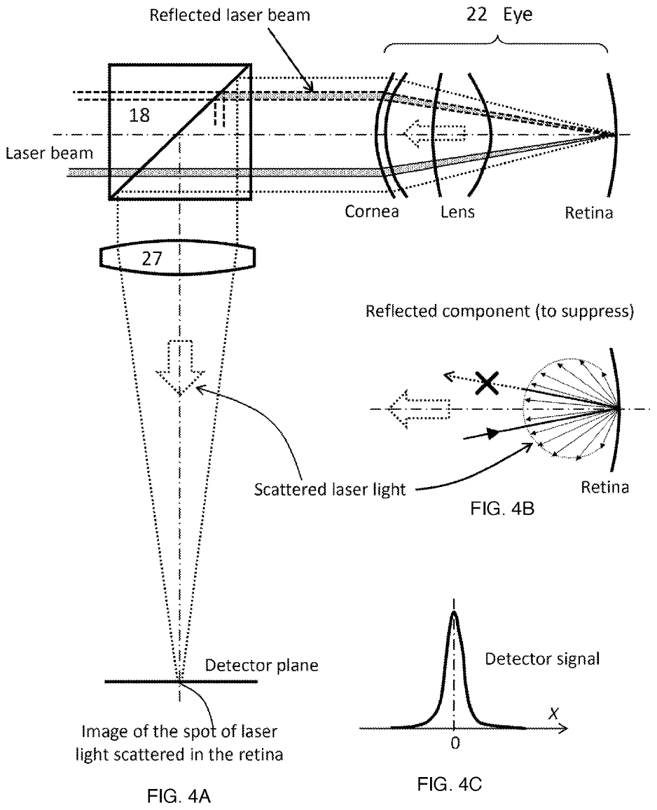

The path to the first detector for the case of an emmetropic eye is shown in FIG. 4A. The polarizing beam splitter 18 allows the vertical linear polarization to come through in both directions—to the eye, and out of the eye. Due to multiple acts of scatter in the retinal tissue, the light coming back from the eye is depolarized, i.e., it contains an orthogonal (horizontal) component, that is reflected by the polarizing beam splitter in the direction of the first detector 28. In FIG. 4A, the conjugating telescope 19 is not shown. In FIG. 4B, the reflected (vertical) component is shown to be suppressed, only the scattered light containing the horizontal component is used for measurement. The shape of light distribution in the photosensitive plane of the detector 28 is shown in FIG. 4C.

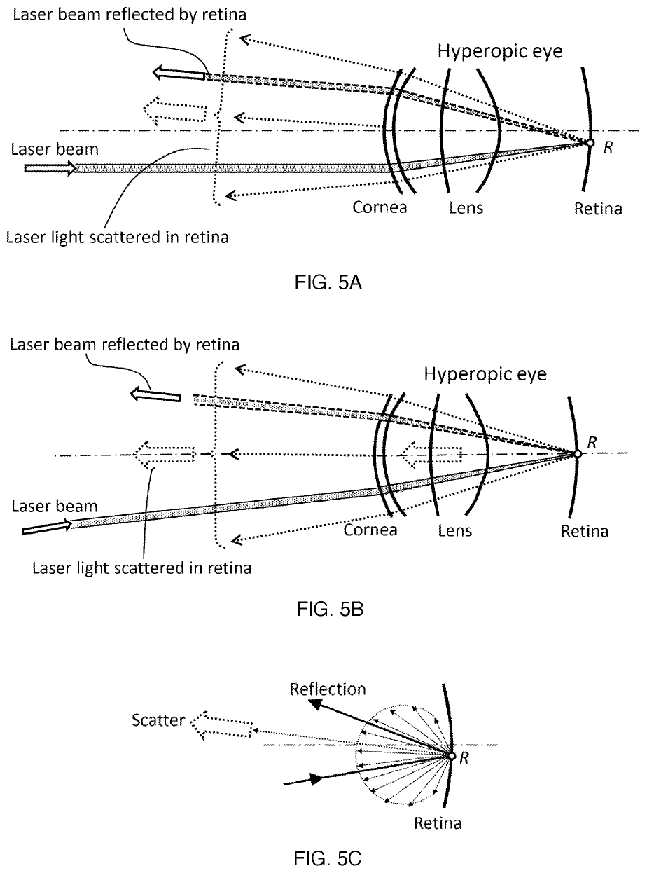

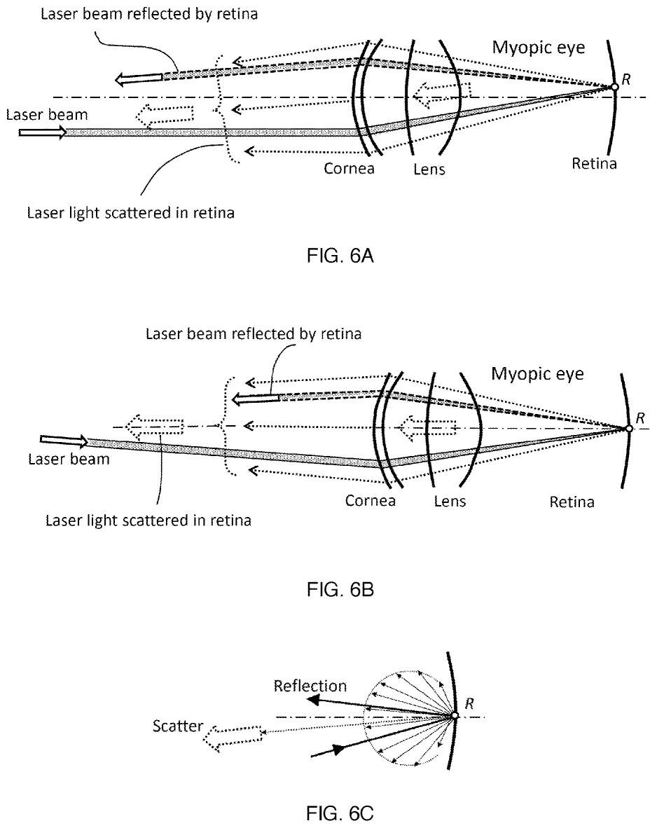

The cases of hyperopic and myopic eyes are demonstrated by FIGS. 5A-5C and FIGS. 6A-6C correspondingly. FIG. 5A and FIG. 6A show the probing of the eye by the laser beam parallel to the optical axis. In both cases, the light coming out of the eye is tilted relatively to the optical axis. As illustrated in FIG. 5B and FIG. 6B, this tilt is compensated by tilting the probing laser beam by the angle corresponding to the defocus component of the Zernike decomposition describing the wave front reconstructed from the data acquired when measurement was performed with the rays parallel to the optical axis. As a result, the defocus is zeroed in the wave front map reconstructed with this laser tilt. Moreover, with such configuration, the image of the laser spot becomes focused in the plane of the first detector. The full conjugation provides both, the adjustment of the tilt of the probing laser beam and the focusing of the image of the laser spot seen by the detector on retina. A powerful feature of full conjugation exploited in the present invention is hitting the eye by each of the laser beams in the same entrance point independently of its tilt, i.e., the n-th laser beam hits the n-th entrance point independently of its tilt. The principle of conjugation for ray tracing technique was described in the US Patent Publication No. 2010/0271595, the entirety of which is hereby incorporated by reference. The versions of its embodiment using the fluidic lenses are described in the Ukrainian Patent Nos. 104397 (V. Molebny, et al. Fast ray tracing spatially resolved refractometer) and 104398 (V. Molebny, et al. Device for measurement of wave front aberrations), the entireties of both of which are hereby incorporated by reference. The conjugation telescope 19 is configured as a 1:1 telescope for an emmetropic eye. Its optimization for the range of the to be measured ametropy must consider an actual course of rays at their different heights, taking into account shapes, dimensions and distances of the optical elements.

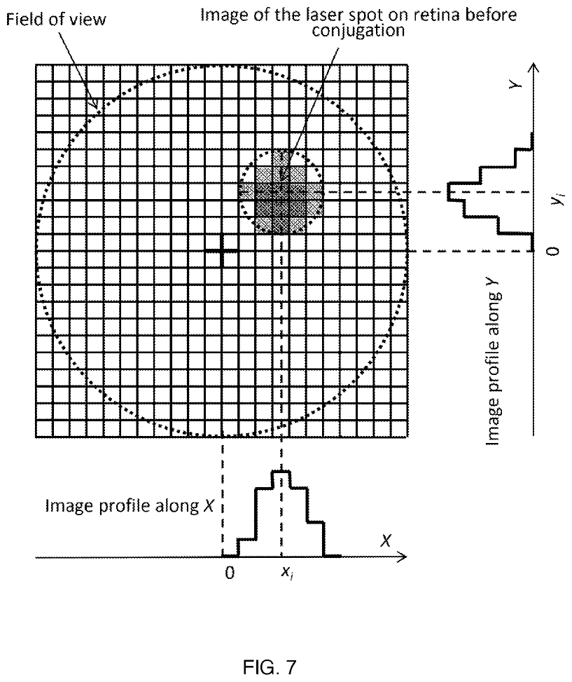

Projection of the image of the laser spot in the plane of the first detector 28 is shown in FIG. 7 for the established field of view. Several types of photosensitive detectors, measuring the position/coordinates, may be used. Among them, there are the position sensing detectors (PSD)—silicon photodiodes providing an analog output directly proportional to the position of a light spot in the detector plane. Theoretically, the two-dimensional CCD matrices could be a better solution, but they do not provide a high-speed redout of data restricted by several hundreds of frames per second. Another solution is based on linear arrays of photodetectors arranged to measure the intensity distribution along X and Y coordinates separately. Their drawback is in doubling the amount of optical components due to the necessity of a two-channel detection. A solution was found by Hamamatsu who arranged X rows and Y columns in a single matrix creating a two-dimensional profile sensor providing the readout of digital information from a single matrix (hamamatsu.com/content/dam/hamamatsu photonics/sites/documents/99_

SALES_ LIBRARY/ssd/s15366_series_kmpd1234e.pdf). Therefore, for the goals of this invention, a preferred type of the photosensitive detector may be the two-dimensional profile sensing detector, for example, a Hamamatsu profile sensor of S15366 series, that is a CMOS area sensor acquiring the projection data with high-speed frame acquisition and readout rate (1602 frames/s with S15366-512, or 3156 frames/s with S15366-256). It has a digital video output, allowing external signal processing via a connection to the processing unit 31 via the bus 33. A simplified presentation of the laser spot image and of its X and Y projections is shown in FIG. 7 for the case before conjugation. The centers of gravity of the X and Y profiles are digitally read out from the first detector 28 and sent to the processing unit 31 via the bus 33.

With measured coordinates (xᵢ, yᵢ) for each projection, a standard ray tracing procedure is applied, and the wave front is reconstructed. The defocus component of the aberration errors is derived from Zernike decomposition and is applied to the fluidic lens 20 of the conjugation telescope 19 via the bus 33 from the processing unit 31.

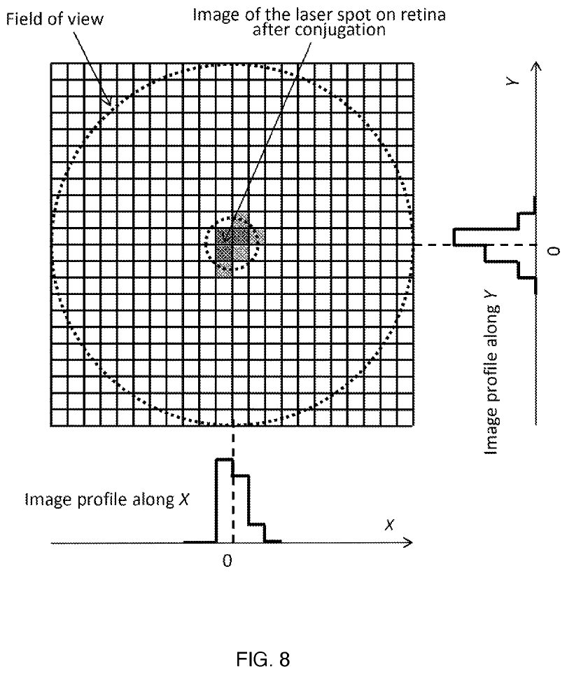

As soon as the fluidic lens 20 has finished accomplishing the command of conjugation for the telescope 19, the next procedure of ray tracing starts. FIG. 8 illustrates what changes take place when the probing is made through telescope 19 in the conjugated position. Firstly, the image of the spot is moved to the center due to the compensated tilt of the probing laser beam. Secondly, the size of the spot image shrinks due to focusing (conjugation telescope 19 provides a parallel course of rays arriving to the lens 27).

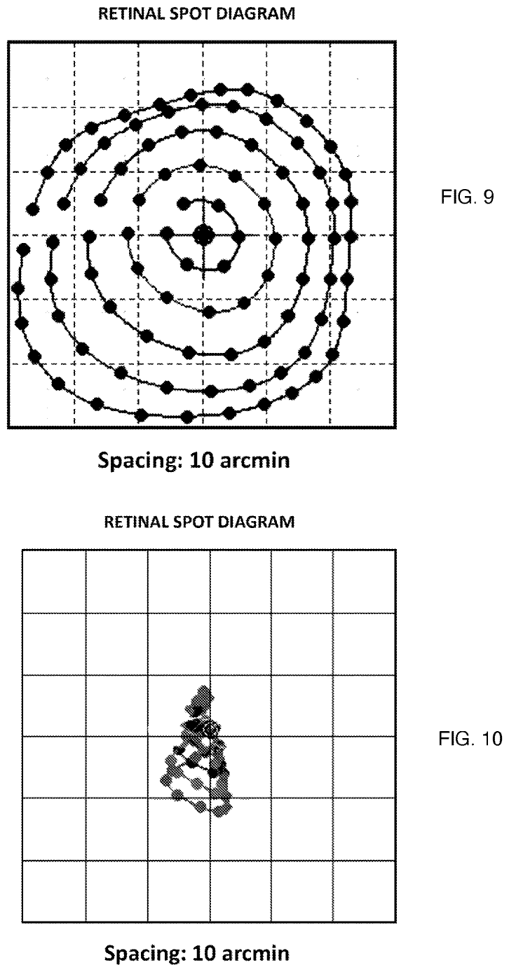

The whole set of the images of retina spots (the retinal spot diagram) accumulated during the complete cycle of point-by-point probing is shown in FIG. 9. The retinal spot diagram shows a near to concentric circle-like configuration of the diagram in the center with small distortions at the periphery. After the conjugation, the retinal spot diagram looks more compact (FIG. 10) with the center of the diagram being an overlay of points representing the central part of the laser beam projections. In FIG. 10, the peripheral part forms a coma-like residue (it can be of any other shape) saying that the eye has the aberrations that can't be compensated for by spherical optics. It is important for the diagnosing and further treatment to know what part of the optical system of the eye is the origin of these residual aberrations. If the origin is the crystalline lens, and the residue is higher than a tolerated level, than the only replacement of the crystalline lens is the solution. Since the amount of the residue can progress with age, it is important to define the in-time moment for the surgery.

The angle of tilt of the laser beam and all data on the measured wave front in the second cycle of measurement (conjugated position of the telescope 19) are written in the memory of the processing unit 31 for further calculations.

Another factor for evaluation of the quality of vision is the scatter of light in the crystalline lens. The present invention solves the problem of the timely determination of the concurrent impact of both factors at the moment when the surgical solution is necessary.

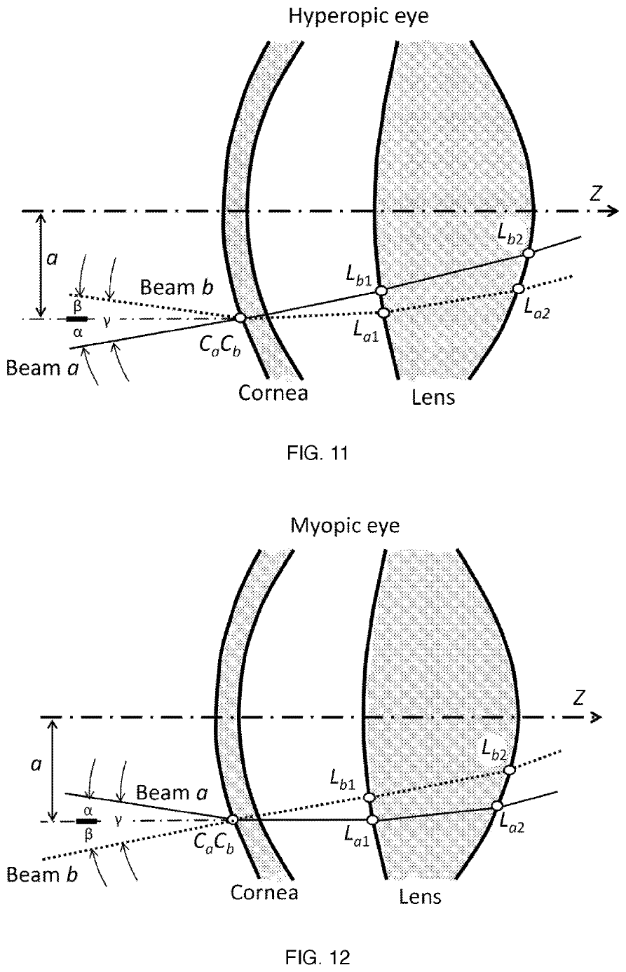

Taking the tilt α of the laser beam in the second cycle of measurement for a starting position (FIG. 11 and FIG. 12), it is necessary to tilt the laser beam in the third cycle of measurement by an angle β relative to the first beam orientation to get a given normalization angle γ between the tilts in the second and the third cycles of measurement. The angles α, β, and γ are in the plane of the optical axis, measured relative to the direction of the laser beam in the first cycle of measurement, i.e., parallel to the optical axis. The operations of tilt control are exercised by the telescope 19 through a program led by the processing unit 31 via the bus 3, or manually. It is expedient to have the normalization, as ratioed to a unit of optical power change, i.e., divided by a value defined by a normalization angle γ=(α+β), shown in FIG. 11 for a hyperopic eye, and in FIG. 12 for a myopic eye. It is expedient to express the normalization value in diopters. Then, the refraction non-homogeneity may be designated in non-dimensional units.

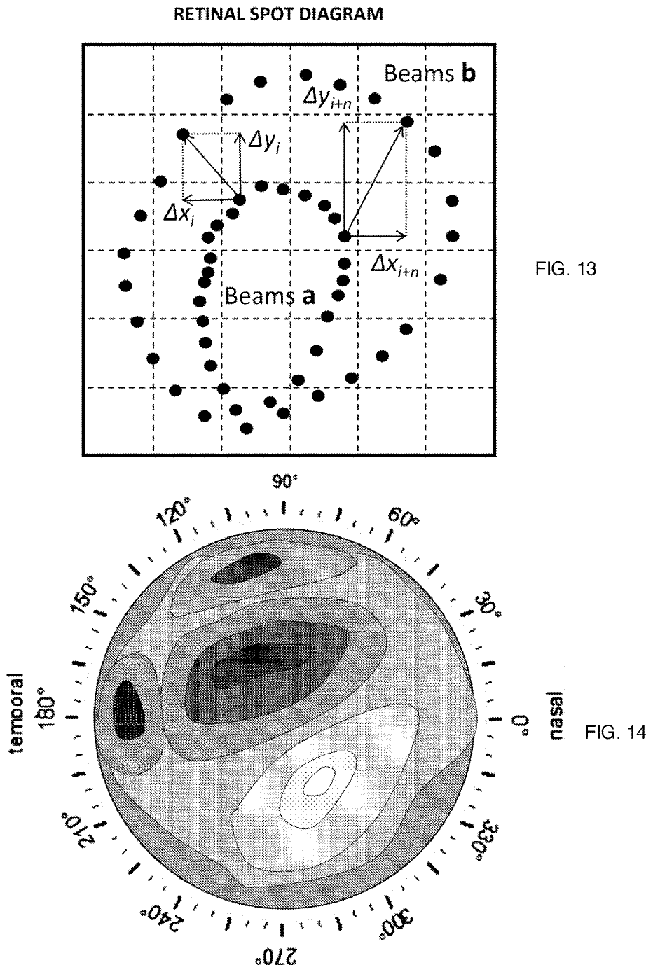

Ukraine Patent No. 122709 teaches that the points of crossing the cornea by the beams a (Cₐ) in the second cycle of measurement and b (C_{b}) in the third cycle of measurement coincide. Neglecting the difference in the paths of crossing the cornea having the thickness of about 0.5 mm, and suggesting that the other eye media are homogeneous, it is evident that the refraction non-homogeneity will be revealed due to the beams a and b following through different sections of the crystalline lens. It means that the retina spot diagrams (FIG. 13) for the beam a and for the beam b will manifest the difference of refraction parameters along the path (Lₐ₁-Lₐ₂) and along the path (L_{b1}-L_{b2}) (FIG. 12). This difference between refraction parameters is measured by the processing unit 31 either in a rectangular, or in a polar system of coordinates.

FIG. 13 illustrates the point-by-point difference in the rectangular system of coordinates. Beams a form an internal point circle corresponding to the conjugated configuration (for the sake of illustration, the spots a are dispersed out of the center). For example, beam a and beam b tilted at an angles α and β correspondingly, having crossed the same i-th point in the cornea, make their traces on the retina with coordinate difference Δₓᵢ and Δ_{yi}. These values are accepted by the processing unit for reconstruction of the wave front map and its normalization by the known difference of tilts γ of beam a and beam b. An example of the wave front difference map is shown in FIG. 14. From these data, the modulation transfer function is calculated and used for evaluation of the quality of the crystalline lens. There are several ways of doing so are described elsewhere.

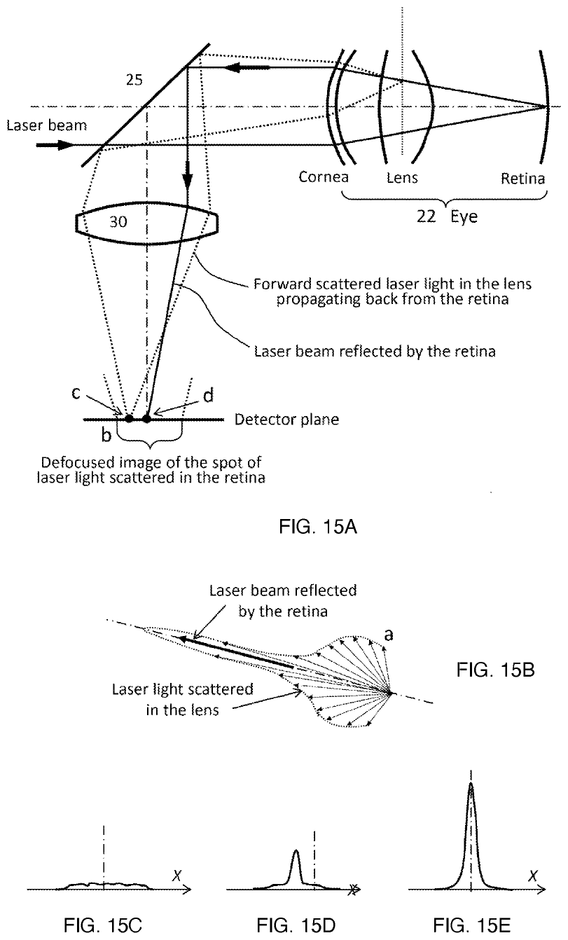

The second detector 29 functions in a similar way with the first detector 28. The laser beam reflected from the retina surface exits the eye, and after reflection from the beam splitter 25 (FIG. 1 and FIG. 15A), propagates to the focal plane of the objective lens 30 coinciding with the photosensitive plane of the second detector 29. The objective lens 30 is positioned in a way to conjugate the planes of the crystalline lens and the plane of the photosensitive surface of the detector 29. Practically, the plane of the crystalline lens should be regarded as its middle cross-section.

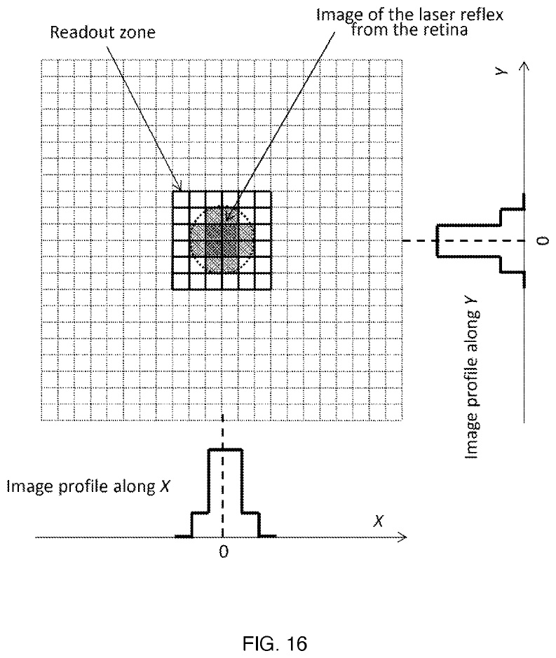

FIG. 15A demonstrates three main phenomena important for this invention. The laser beam reflected from the retina is shown in a bold line. The track of the light scattered in the crystalline lens is shown as a bunch, confined by dotted lines. FIG. 15B shows the all-directional character of the scatter of the light in the process of crossing the lens by the reflected laser beam. The diagram of scatter (indicatrix) has a shape elongated in the direction of beam propagation. As a result, three kinds of light hit the photosensitive surface of the detector 29: (1) light scattered by the tissues of the retina (FIG. 15C); (2) light scattered in the crystalline lens along the trace of the reflected laser beam (FIG. 15D); (3) light of the reflected laser beam (FIG. 15E). Its position in the plane of the detector 29 in the case of emmetropic eye is in the center of the coordinate system (FIG. 16). Normally, it results in the higher level of the signal as compared with the signal from the detector 28. But in several positions of the probing beam, it can be overlaid by the Purkinje reflections, that in the case of detector 28, are suppressed by the polarization selection. Another way to avoid the corruption in the restoration of the wave front data, is to exclude the data on these points from further calculations.

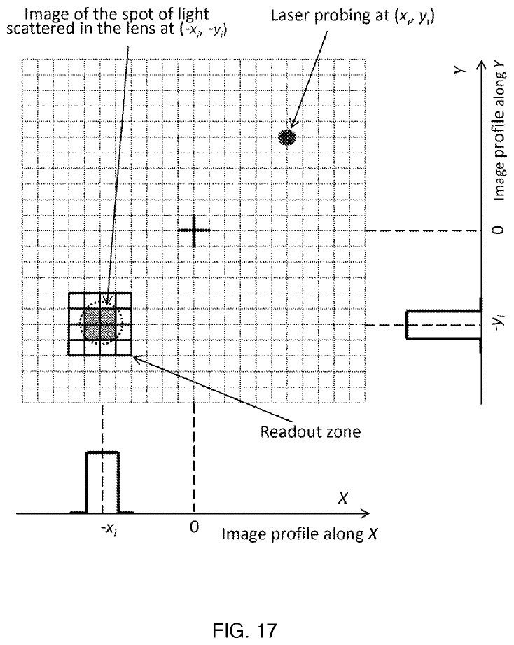

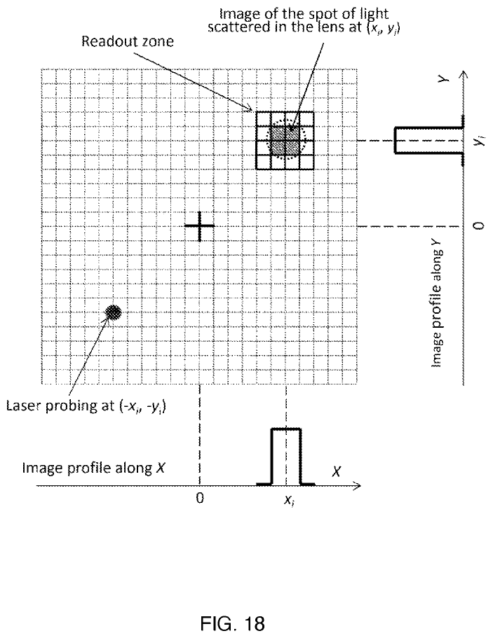

Light scattered by the retina is far from the focus, and there why it forms a background splashed over the whole field of view (FIG. 15C). Light scattered by the crystalline lens forms a spot representing the whole depth of the path of the reflected laser beam in the crystalline lens. Its position in the focal plane of the detector 29 corresponds to the coordinates of the beam crossing the lens (FIG. 17). Because the probing laser beam is in the same plane with the optical axis of the instrument, the zone of exit of the reflected laser beam is located at the symmetrical coordinates with the entrance point of the probing laser beam. For example, in the case of the emmetropic eye, if the point of beam entrance has the coordinates (xᵢ, yᵢ), then the coordinates of the zone of exit should be (−xᵢ, −yᵢ). The reverse situation is shown in FIG. 18 with the entrance point in (−xᵢ, −yᵢ), and the exit zone in (xᵢ, yᵢ). Using this feature, the readout is made of the spot image of the scatter in the lens in each probing point (xᵢ, yᵢ) with the data addressed to the symmetric point (−xᵢ, −yᵢ).

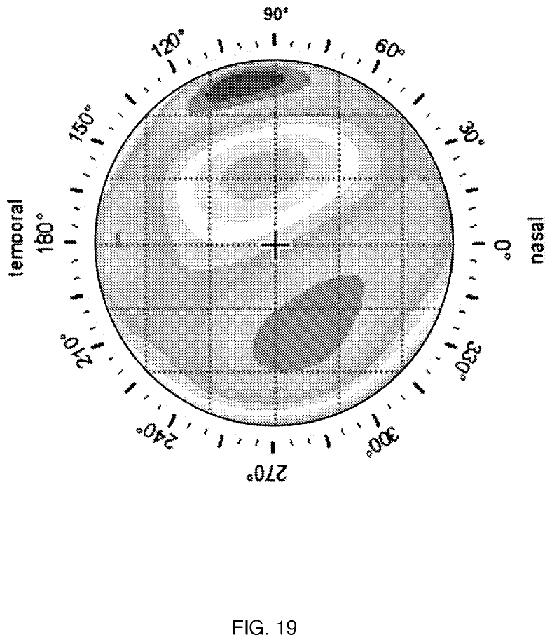

The sequence of events at measuring the scatter properties of the crystalline lens with the detector 29 is as follows. For each (xᵢ, yᵢ) point of probing, a readout is made of the returned light intensity in two points—the first one corresponding to the reflected laser beam, and the second one corresponding to the scatter in the lens in the zone with the coordinates (−xᵢ, −yᵢ). As mentioned, these data are related to the coordinates (−xᵢ, −yᵢ), and therefore are to be kept in the memory of the processing unit 31 until probing the point (−xᵢ, −yᵢ). Then, the ratio for the intensity of the scattered light to the intensity of the reflected light is calculated in each point, and a map of this ratio distribution is reconstructed. An example of such distribution represented as a diagram of a polynomial description is shown in FIG. 19. For a single number description, the data on scatter are averaged over the aperture and presented on the display.

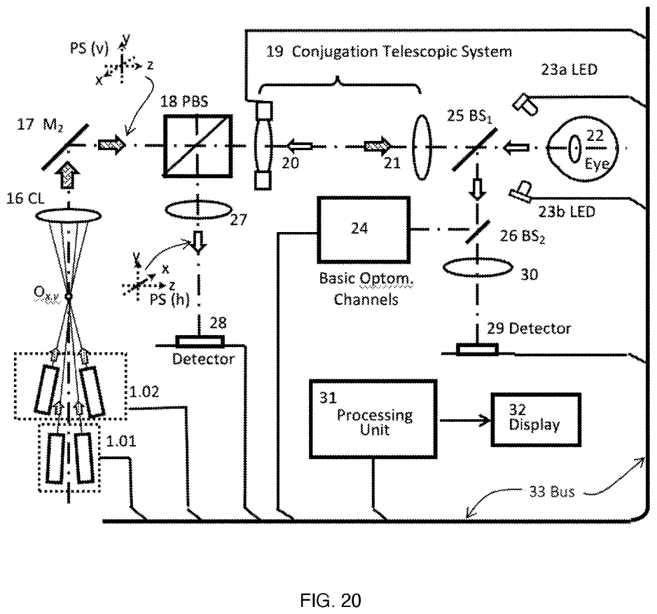

With continued reference to FIG. 1, FIG. 20 illustrates a device that utilizes a plurality of lasers 1.01, 1.02 which replaces the single laser 1, the two-coordinate deflector 2, the first mirror 11, the telescope 12, and the aperture 15. The beam paths from the lasers 1.01, 1.02 are collimated by the collimating lens 16 with a front focus O_{x,y} and then proceeds along a path as in FIG. 1 to and from the eye/crystalline lens 22 via components 17 to 21 and 23a,b to 30. The device is electrically controlled by a processing unit 31 through the bus 33. The processing unit 31 is connected to a displaying means 32 via the bus 33.

Still another outcome of the measurement is the evaluation of the concurrent impact of refraction non-homogeneity and scatter in the lens on the quality of vision. The estimate is provided by calculation in the processing unit 31 of the correlation between the distribution of refraction non-homogeneity of the crystalline lens and of the scatter in the lens. A higher correlation means that the scatter shadows steeper variations of the refraction, thus making their impact weaker, and thereby masking the refraction deficiencies. This estimate may be important for timely decision on surgical treatment.

1: A method for measuring an optical quality of a human eye based on laser ray tracing, the method comprising:
probing the human eye sequentially in time with a laser beam in a set of points within an aperture of the eye,
detecting the projections of the laser beam reflected and scattered from the retina; and
processing the detected images of the laser spots.

2: The method of claim 1, further comprising:
calculating a Strehl ratio for each probing point separately, said calculation made on signal intensity distributions in detected images of the laser spots of laser projections comprising their point spread functions in a status of a compensated defocus of the projected laser beam and of the detected images of said laser spots;
reconstructing a map of the optical quality of the eye with the values of said optical quality in each probing point that is a function of the Strehl ratio; and
calculating a total quality of the eye as a ratio of a number of probing points with a quality higher than the established threshold to the total number of the probing points.

3: A method for measuring an optical quality of a human eye based on laser ray tracing, comprising:
probing the human eye sequentially in time with a linearly polarized laser beam in a set of points within the aperture of the eye;
detecting images of laser spots on a retina with a photosensitive detector;
processing detected signals; and
reconstructing a distribution of wave front errors.

4: The method of claim 3, further comprising:
calculating a defocus component from the reconstructed distribution of wave front errors;
probing the human eye with the linearly polarized laser beam projected therein at an angle compensating for the defocus component;
selecting an orthogonally polarized component of light coming from the human eye,
detecting the orthogonally polarized component in a detection plane conjugated with the retina,
measuring a shape and coordinates of a detected image of a spot of the laser projection on the retina;
calculating a Strehl ratio in each probing point separately for the point spread function described by a measured shape of the spot image of the projected laser beam,
reconstructing a map of the optical quality of the human eye with values of the optical quality in each probing point that is the function of the Strehl ratio, and
calculating a total quality of the eye represented by a ratio of the number of probing points with the quality higher than an established threshold to the total number of the probing points.

5: A method for measuring an optical quality of a crystalline lens of an eye based on laser ray tracing, comprising:
probing the eye sequentially in time with a laser beam in a set of points within an aperture of the eye;
detecting images of laser spots on a retina with a photosensitive detector, processing the detected signals;
reconstructing the distribution of the wave front errors; and
calculating the modulation transfer function.

6: The method of claim 5, further comprising:
calculating a defocus component from the reconstructed distribution of the wave front errors;
probing the eye in a set of probing points over the aperture thereof with a linearly polarized laser beam projected into the eye at a first angle that is an angle compensating for a defocus component;
selecting an orthogonally polarized component of a light coming from the eye;
detecting said orthogonally polarized component in a detection plane conjugated with the retina;
measuring coordinates of the detected image of a spot of a laser projection on the retina;
probing the eye in the same set of probing points over the aperture with the linearly polarized laser beam projected in the eye at a second angle differing from the first angle by a normalization value;
selecting the orthogonally polarized component of the light coming from the eye, detecting said orthogonally polarized component in the detection plane conjugated with the retina;
measuring coordinates of the detected image of a spot of the laser projection on the retina;
calculating s difference between coordinates of the detected spot images at the first angle of the probing laser beam and the second angle of the probing laser beam in the same probing points;
reconstructing a two-dimensional distribution of the wave front difference; and
calculating a ratio of an area of a surface of the two-dimensional distribution having a wave front difference lower than an established threshold of a total area of the surface of said two-dimensional distribution.

7: The method of claim 6, wherein the optical quality of the crystalline lens is a function of the calculated ratio.

8: A method for measuring an optical quality of a crystalline lens of an eye, based on laser ray tracing, the method comprising:
probing the eye sequentially in time with a laser beam in a set of points within an aperture of the eye;
detecting images of the laser spots on a retina with a photosensitive detector;
processing the detected signals; and
reconstructing a distribution of wave front errors;
wherein:
a preliminary probing of the eye in parallel to an optical axis thereof is provided and a defocus component is derived from the reconstructed wave front errors;
said laser beam is linearly polarized and is projected in the eye at an angle compensating for said defocus component;
a first plane of detection is provided and is conjugated with a plane of a retina and a second plane of detection is conjugated with a plane inside the crystalline lens orthogonal to the optical axis;
the light coming from the eye is detected separately for each of a first polarized component and a second polarized component, said first polarized component having a plane of polarization orthogonal to the polarization of the probing laser beam, the second polarized component being in the plane of polarization of said probing laser beam;
in a first plane of conjugation, coordinates are measured of the spot imaged on a two-dimensionally photosensitive detector for the first polarization component, corresponding to the laser beam scattered in the retina, and the two-dimensional distribution of the wave front errors is reconstructed;
in a second plane of conjugation, signal levels are measured of each of a first spot and a second spot imaged on the two-dimensionally photosensitive detector for the second polarization component, said first spot produced by the reflection of the laser beam from the retina and having passed through the crystalline lens in an outward direction, and said second spot being an image of a scattering trace of the outward passing laser beam in the crystalline lens;
a two-dimensional distribution of scatter in the crystalline lens is reconstructed as a ratio of a signal level of the second spot to a signal level of the first spot, and its dimensionless index is calculated as an averaged value of said ratios; and
the optical quality of the crystalline lens scaled in a dimensionless index is calculated as a degradation of a modulation transfer function of the eye without a deducted corneal component, said degradation of the modulation transfer function is referenced to said two-dimensional distribution of scatter in the crystalline lens.

9: The method of claim 8, wherein coordinates of the first spot imaged in the second plane of conjugation are measured for the second polarization component, said measurements provided in a central zone of the two-dimensionally photosensitive detector such that signals outside said central zone are excluded from measurements, and a size of said central zone is restricted by a size of a cross-section of the reflected laser beam and a preassigned range of dispersion of spot coordinates.

10: The method of claim 9, wherein the coordinates of said first spot that are a result of ray tracing of the eye through any of the set of probing points are compared with the coordinates of another first spot that are the result of ray tracing of the eye through the probing point positioned in an aperture of the eye symmetrically with the optical axis; and
a differential modulation transfer function is calculated from a difference between the compared spot coordinates, and the optical quality of the crystalline lens scaled in a dimensionless index is calculated as a degradation of said differential modulation transfer function as referenced to said two-dimensional distribution of scatter in the crystalline lens.

11: The method of claim 9, wherein the coordinates of said second spot imaged in the second plane of conjugation are measured for the second polarization component, the measurements provided in a central zone with the coordinates of its center symmetrical with the coordinates of eye probing by the laser beam in regard to the optical axis, the signals outside said zone excluded from measurements, and a size of said zone restricted by a size of a cross-section of the reflected laser beam and a preinstalled range of the spot coordinates dispersion.

12: The method of claim 9, wherein the coordinates of said first spot imaged in the second plane of conjugation are measured for the second polarization component, said measurements provided in an annular zone, whose internal size is restricted by a zone of Purkinje reflections;
wave front errors are calculated with deduction of the corneal component in said annular zone, and compared with the wave front errors in said annular zone calculated from the measurements of the spot coordinates imaged in the first plane of conjugation for the first polarization component; and
a differential modulation transfer function is calculated for said annular zone, and the optical quality of the crystalline lens scaled in a dimensionless index is calculated as the degradation of differential modulation transfer function, being referenced to said two-dimensional distribution of scatter in said annular zone.

13: The method of claim 9, wherein a value of any of the signals partially or completely created by the second polarization component exceeding a preinstalled level regarding an average value, and its exclusion from calculations of the optical quality of the crystalline lens is evaluated.

14: A device for measurement of the optical quality of the crystalline lens of the human eye, comprising:
a laser configured to emit a laser beam of a wavelength suitable for propagation in the human eye;
a two-axis acousto-optical deflector on a path of the laser beam;
a two-output generator of digitally synthesized sinusoidal voltages with its outputs electrically connected to said two-axial acousto-optical deflector and configured to control the deflection of said laser beam in both orthogonal directions;
a collimating optical system placed at the exit of the two-axis acousto-optical deflector with its front focus in a center of beam deflection;
a conjugating telescopic system with a lens component or a mirror component and with its optical axis coincident with an optical axis of said collimating optical system;
a polarizing beam splitter placed between said collimating optical system and said conjugating telescopic system;
a first photosensitive detector with a coordinate measurement function placed in the path of the laser light reflected by said polarizing beam splitter with the feature of optical conjugation with the plane of the retina;
a second photosensitive detector with a profile measurement function is placed through a beam splitter installed between the eye and the lens component or the mirror component of said conjugating telescope system, said profile measuring photosensitive detector is optically conjugated with the plane inside the crystalline lens orthogonal to the optical axis; and
a processing unit in operable communication with each of the laser, the two-output generator of digitally synthesized sinusoidal voltages, the conjugating telescopic system, and the position sensitive detection system.

15: A device for measurement of the optical quality of the crystalline lens of the human eye, comprising:
a multi-laser block containing a plurality of lasers, each of said plurality configured to emit independently a laser beam of a wavelength suitable for propagation in the human eye, an optical axis of each laser in the plurality oriented to cross an optical axis of the device in a single point;
a collimating optical system placed on the optical axis of the device with its front focus in a center of beam deflection;
a conjugating telescopic system with a lens component or a mirror component having an optical axis coincident with an optical axis of the collimating optical system;
a polarizing beam splitter placed between the collimating optical system and the conjugating telescopic system;
a first photosensitive detector with a coordinate measurement function placed in a path of laser light reflected by said polarizing beam splitter with a feature of optical conjugation with a plane of a retina in the human eye;
a second photosensitive detector with a profile measurement function is placed through a beam splitter positioned between the human eye and the lens component or the mirror component of the conjugating telescope system, the profile measuring photosensitive detector is optically conjugated with the plane inside the crystalline lens orthogonal to the optical axis; and
a processing unit in operable communication with each of the plurality of lasers, a two-output generator of digitally synthesized sinusoidal voltages, the conjugating telescopic system, and a position sensitive detection system.

16: The device of claim 15, wherein the conjugating telescopic system comprises at least one electrically controlled element with a variable optical power configured to compensate for an ametropy of the eye.

17: The device of claim 15, wherein the optical conjugation of the photosensitive plane of said coordinate measuring photosensitive detector is via the conjugating telescopic system, said optical conjugation adaptive to the optical power of the eye, said adaptation provided by at least one of the lens component or the mirror component of the conjugating telescopic system that is configured to control the optical power of both of the lens component and the mirror component via communication with said processing unit.

18: The device of claim 15, wherein the polarizing beam splitter is configured to suppress the component of light polarized in the plane of polarization of the laser beam returning from the eye in the direction to the coordinate measuring photosensitive detector.

19: The device of claim 15, wherein the second detector channel is configured to read-off the information from the restricted zone of the photosensitive area, said restricted zone controlled through communication with the processing unit.
